(19) United States
(12) Patent Application Publication
(10) Pub. No.: US 2004/0009992 A1
(43) Pub. Date: Jan. 15, 2004

(54) Azolylalkylazole derivatives, their preparation, and their use as pesticides

(76) Inventors: Wolfgang Schaper, Diedorf (DE); Henricus Maria Martinus, Usingen (DE); Sven Harmsen, Lubeck (DE); Uwe Doller, Rodgau (DE); Jorg Tiebes, Frankfurt (DE); Daniela Jans, Bad Homburg v. d. H. (DE); Waltraud Hempel, Liederbach (DE); Ulrich Sanft, Eppstein/Ts. (DE); Maria-Theresia Thonessen, Heidesheim (DE)

Correspondence Address: FROMMER LAWRENCE & HAUG, 745 FIFTH AVENUE- 10TH FL., NEW YORK, NY, 10151, US

(21) Appl. No.: 10/463,252

(22) Filed: Jun. 17, 2003

Related U.S. Application Data
(63) Continuation of application No. 09/746,111, filed on Dec. 21, 2000, now abandoned.

(30) Foreign Application Priority Data
Dec. 23, 1999 (DE) 19962901.3

(51) Int. Cl. C07D 413/14; A01N 43/54; A01N 43/40

(52) U.S. Cl. 514/256; 514/341; 544/333; 546/269.4

(57) ABSTRACT

Azolylalkyloxazole and -oxadiazole derivatives of the formula (I)

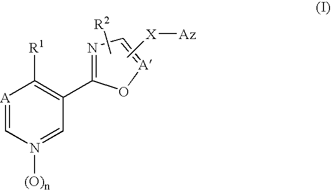

in which the symbols and indices are as defined in the description are highly suitable for controlling animal pests such as insects, arachnids, nematodes, helminths and molluscs and for controlling endo- and ectoparasites in the field of veterinary medicine while being well tolerated by plants and having a favorable toxicity to warm-blooded species.

The invention relates to azolylalkylazole derivatives, to their preparation, and to their use for controlling animal pests, in particular insects, spider mites, ectoparasites and helminths.

It has already been disclosed that certain heterocyclylalkylazoles, in particular 3-[2-(pyrrol-1-yl)ethyl]-5-(4-trifluoromethyl-3-pyridyl)-1,2,4-oxadiazole, exhibit an insecticidal action (WO-A 98/57969). However, the biological action of these compounds is not satisfactory in all fields of application, in particular when low application rates and concentrations are used.

There have now been found azolylalkyloxazole and -oxadiazole derivatives of the formula (I)

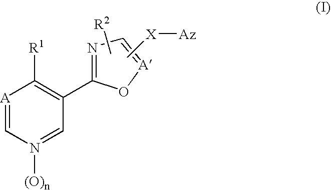

where the symbols and indices are as defined below,

which are highly suitable for controlling animal pests such as insects and arachnids and for controlling ectoparasites in the field of veterinary medicine while being well tolerated by plants and having a favorable toxicity to warm-blooded species.

The invention therefore relates to compounds of the formula (I) in which the symbols and indices are as defined below:

R¹ is (C₁-C₄)haloalkyl, in particular (C₁-C₄)fluoroalkyl;

R² is hydrogen, halogen, (C₁-C₄)alkyl, (C₃-C₈)cycloalkyl or (C₁-C₄)haloalkyl;

A,A′ are identical or different and are in each case CH or N;

n is 0 or 1;

X is a branched or unbranched (C₁-C₈)alkylene unit in which a C—C single bond can optionally be replaced by a double or triple bond and in which furthermore one or more CH₂ groups can be replaced by a carbonyl group or a heteroatom unit such as oxygen, S(O)ₓ where x=0, 1 or 2, NR³, where R³=H, (C₁-C₄)alkyl, C₃- or C₄-alkenyl or -alkynyl, (C₁-C₄)alkylsulfonyl, (C₁-C₄)alkanoyl, (C₁-C₄)alkoxycarbonyl, or

dimethylsilyl, and where additionally 3 to 8 atoms of this hydrocarbon radical, which is optionally modified as above, may form a cycle;

Az is a group of the formula (II)

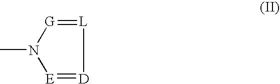

where the symbols have the following meanings:

E, D, G, L are identical or different and are CH or N, where in each case at least one of the symbols E, D, G and L is CH and at least one is N

and where carbon atoms are optionally substituted and the substituents of adjacent carbon atoms, optionally together with the carbon atoms of the group Az, can form a ring.

The symbols and indices in the formula (I) preferably have the following meanings:

R¹ is preferably (C₁-C₄)fluoroalkyl, in particular trifluoromethyl.

R² is preferably hydrogen or methyl, in particular hydrogen.

n is preferably 0.

A is preferably CH.

A′ is preferably N.

X is preferably (C₁-C₄)alkylene, in particular —(CH₂)—, —(CH₂)₂— or —(CH₂)₃—.

Az is preferably an imidazolyl, pyrazolyl or triazolyl radical which can be substituted by one, two or three radicals, especially preferably the imidazolyl, pyrazolyl or the 1,2,4-triazol-1-yl radical, in particular the pyrazolyl or the 1,2,4-triazol-1-yl radical.

Preferred identical or different radicals (R⁴) by which Az can be substituted are:

a) halogen, hydroxyl, cyano, thiocyano, nitro;

b) (C₁-C₈)alkyl, (C₂-C₈)alkenyl, (C₂-C₈)alkynyl, (C₃-C₈)cycloalkyl, (C₄-C₈)cycloalkenyl, aryl, heterocyclyl, (C₁-C₈)alkoxy, (C₃-C₈)alkenyloxy, (C₃-C₈)alkynyloxy, (C₁-C₈)alkylthio, (C₁-C₈)alkylsulfinyl, (C₁-C₈)alkylsulfonyl, (C₂-C₈)alkenylthio, (C₂-C₈)alkynylthio, —COY, where Y is hydrogen, (C₁-C₈)alkyl, (C₁-C₈)alkoxy, amino, (C₁-C₈)alkylamino or (C₁-C₈)dialkylamino, or C(NOR⁵)W where R⁵ is hydrogen, (C₁-C₈)alkyl, aryl(C₁-C₄)alkyl or aryl and W is hydrogen, (C₁-C₈)alkyl or aryl, or C(NNR⁶R⁷) where R⁶ and R⁷ are identical or different and are hydrogen, (C₁-C₈)alkyl, (C₁-C₈)alkanoyl, (C₁-C₈)alkoxycarbonyl or aryl, or C(O(CH₂)_{1,2,3}O) where the alkylenedioxy unit can be substituted by up to four (C₁-C₄)alkyl groups, or —NZ¹Z² where Z¹ is hydrogen or (C₁-C₈)alkyl and Z² is (C₁-C₈)alkanoyl, (C₁-C₈)alkoxycarbonyl, (C₁-C₈)alkyl, (C₁-C₄)alkylsulfonyl or hydrogen; or (C₁-C₈)trialkylsilyl,

where a saturated carbon unit in the various R⁴ alkyl, cycloalkyl, alkenyl, alkynyl, cycloalkenyl radicals or the groups derived therefrom, such as the alkoxy, alkenyloxy, alkynyloxy, alkylthio, alkylsulfinyl, alkylsulfonyl, alkenylthio, alkynylthio, alkylamino, dialkylamino, trialkylsilyl, alkanoyl or alkoxycarbonyl group, can optionally be replaced by a carbonyl group or a heteroatom unit such as oxygen, S(O)ₓ where x=0, 1 or 2, or dimethylsilyl, and where additionally 3 to 8 atoms of these hydrocarbon radicals, which are optionally modified as above, may form a cycle and these hydrocarbon radicals, with or without the stated variations, are optionally substituted by one or more, preferably one to three, in the case of fluorine up to the maximum number of, identical or different radicals selected from the series consisting of halogen, aryl, aryloxy, arylthio, cycloalkoxy, cycloalkylthio, heterocyclyl, heterocyclyloxy, heterocyclylthio, alkanoyl, cycloalkanoyl, haloalkanoyl, aroyl, arylalkanoyl, cycloalkylalkanoyl, alkoxycarbonyl, haloalkoxycarbonyl, cycloalkoxycarbonyl, cycloalkylalkoxycarbonyl, arylalkoxycarbonyl, heterocyclylalkoxycarbonyl, aryloxycarbonyl, heterocyclyloxycarbonyl, alkanoyloxy, haloalkanoyloxy, cycloalkanoyloxy, cycloalkylalkanoyloxy, aryloxy, arylalkanoyloxy, heterocycloylalkanoyloxy, alkylsulfonyloxy, arylsulfonyloxy, hydroxyl, cyano, thiocyano or nitro, and

where the R⁴ cycloaliphatic, aromatic or heterocyclic ring systems are unsubstituted or optionally provided with up to three, in the case of fluorine also up to the maximum number of, identical or different substituents, preferably selected from the group consisting of halogen, nitro, cyano, di-(C₁-C₄)alkylamino, (C₁-C₄)alkyl, (C₃-C₈)cycloalkyl, (C₁-C₄)trialkylsilyl, (C₁-C₄)alkoxy, (C₁-C₄)alkoxy-(C₁-C₄)alkyl, (C₁-C₂)alkoxy-[CH₂CH₂O]_{0,1,2}-ethoxy, (C₁-C₄)alkylthio, (C₁-C₄)alkylsulfinyl, (C₁-C₄)alkylsulfonyl, it being possible for one or more, in the case of fluorine also up to the maximum number of, hydrogen atoms in the alkyl radicals and the radicals derived therefrom to be replaced by halogen, preferably chlorine or fluorine, where, in the event that these substituents are (C₁-C₄)alkyl, these may also be linked cyclically and one or two aliphatic carbon units in these fused ring systems, such as, for example, the indane, di-, tetra- or decahydronaphthyl or benzocycloheptane system, can be replaced by heteroatom units such as oxygen or sulfur, and one or more, in the case of fluorine also up to the maximum number of, hydrogen atoms on the aliphatic carbon atom units can be replaced by halogen or (C₁-C₄)alkyl;

c) two radicals R⁴ which are bonded to adjacent carbon atoms (D, E; L, D or G, L) together with these carbon atoms form an unsaturated 5- or 6-membered carbocyclic ring which, if it takes the form of a 5-membered ring, may contain an oxygen or sulfur atom in place of CH₂ or, if it takes the form of a 6-membered ring, may contain one or two nitrogen atoms in place of one or two CH units, and which is optionally substituted by 1, 2 or 3 identical or different radicals, these radicals denoting (C₁-C₄)alkyl, (C₁-C₄)haloalkyl, (C₁-C₄)alkoxy or hydroxyl, preferably methyl, trifluoromethyl, halogen, preferably fluorine or chlorine, or methoxy, where, in the event that it takes the form of a nitrogen heterocycle and the substituent(s) take(s) the form of hydroxyl groups, the ring system may also exist in the tautomeric keto form, in which case a (C₁-C₄)alkyl substituent which may be present may also be attached to a nitrogen atom, or

d) two radicals R⁴ together with the carbon atoms (GL, LD, DE) to which they are bonded form a saturated 5-, 6- or 7-membered carbocyclic ring which is optionally substituted by 1, 2 or 3 (C₁-C₄)alkyl groups.

Especially preferably,

R⁴ is hydrogen, (C₁-C₄)alkyl, in particular methyl, trifluoromethyl, fluorine, chlorine, bromine, cyano or nitro, or two radicals R⁴ are linked to give a benzo fusion, very especially preferably R⁴ is hydrogen, methyl, chlorine, bromine, cyano, nitro or trifluoromethyl.

Thus, preferred compounds of the formula (I) are those in which

R¹ is fluoroalkyl, in particular trifluoromethyl,

R² is hydrogen or methyl,

n is 0 and

A is CH.

More preferred compounds of the formula (I) are those in which

R¹ is trifluoromethyl,

R² is hydrogen,

n is 0 and

A is CH.

Even more preferred compounds of the formula (I) are those in which

R¹ is trifluoromethyl,

n is 0,

A is CH and

A′ is nitrogen.

Furthermore preferred compounds are those in which X is (C₁-C₄)alkylene, preferably unbranched (C₁-C₃)alkylene.

Furthermore preferred compounds of the formula (I) are those in which

Az is an imidazolyl, pyrazolyl or 1,2,4-triazol-1-yl radical and, if appropriate,

R⁴ is (C₁-C₄)alkyl, in particular methyl, trifluoromethyl, fluorine, chlorine, bromine, cyano or nitro, or two radicals R⁴ are linked to give a benzo fusion.

More preferred are furthermore those compounds of the formula (I) in which

Az is a pyrazolyl or 1,2,4-triazol-1-yl radical and, if appropriate,

R⁴ is methyl, chlorine, bromine, cyano, nitro or trifluoromethyl.

In the above formula, “halogen” is to be understood as meaning a fluorine, chlorine, bromine or iodine atom;

the term “(C₁-C₄)alkyl” is to be understood as meaning an unbranched or branched hydrocarbon radical having 1 to 4 carbon atoms, such as, for example, methyl, ethyl, propyl, isopropyl, 1-butyl, 2-butyl, 2-methylpropyl or tert-butyl radical;

the term “(C₁-C₈)alkyl” the abovementioned alkyl radicals and, for example, pentyl, 2-methylbutyl, 1,1-dimethylpropyl, hexyl, heptyl, octyl or the 1,1,3,3-tetramethylbutyl radical;

the term “(C₁-C₄)haloalkyl” an alkyl group mentioned under the term “(C₁-C₄)alkyl” in which one or more hydrogen atoms are replaced by the abovementioned halogen atoms, preferably chlorine or fluorine, such as, for example, the trifluoromethyl group, 1-fluoroethyl group, the 2,2,2-trifluoroethyl group, the chloromethyl group, the fluoromethyl group, the difluoromethyl group, the 1,1,2,2-tetrafluoroethyl group or the difluorochloromethyl group;

the term “branched or unbranc.hed (C₁-C₈)alkylene unit”, for example, the —(CH₂)—, —(CH₂)₂—, —CH(CH₃)—, —(CH₂)₃—, —CH₂CH(CH₃)—, —(CH₂)₄, —CH₂—CH(CH₃)CH₂—, —(CH₂)₂—CH(CH₃)—, —(CH₂)₅—, —(CH₂)₆—, —(CH₂)₇— or —(CH₂)₈ unit;

the radical Az of the formula (II), for example, the imidazol-1-yl, pyrazol-1-yl, 1,2,4-triazol-1-yl, 1,2,4-triazol-4-yl, 1,2,3-triazol-1-yl, 1,2,3-triazol-2-yl or the 1,2,3,4-tetrazolyl radical.

The terms “alkenyl” and “alkynyl” with a prefix for the range of carbon atoms denote a straight-chain or branched hydrocarbon radical with a number of carbon atoms corresponding to this prefix which contains at least one multiple bond, it being possible for the latter to be located in any position of the unsaturated radical in question.

The term “(C₃-C₈)cycloalkyl” is to be understood as meaning the cyclopropyl, cyclobutyl, cyclopentyl, cyclohexyl, cycloheptyl or cyclooctyl group, the term “aryl” a carbocyclic aromatic radical having preferably 6 to 14, in particular 6 to 12, carbon atoms, for example phenyl, naphthyl or biphenylyl, preferably phenyl; the term “heterocyclyl” preferably a heteroaromatic or heteroaliphatic ring system, “heteroaromatic ring system” preferably being understood as meaning an aryl radical in which at least one CH group is replaced by N and/or at least two adjacent CH groups are replaced by S, NH or O, for example a radical of thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-triazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or 4H-quinolizine;

and the term “heteroaliphatic ring system” preferably a (C₃-C₈)cycloalkyl radical in which at least one carbon unit is replaced by O, S or a group NR⁵ and R⁵ is hydrogen, (C₁-C₄)alkyl, (C₁-C₄)alkanoyl, (C₁-C₄)alkoxycarbonyl, (C₁-C₄)alkylsulfonyl, (C₁-C₄)alkoxy or aryl;

the term “(C₄-C₈)-cycloalkenyl”, for example, the cyclobutenyl, cyclopentenyl, cyclohexenyl, cycloheptenyl or cyclooctenyl group;

the term “(C₁-C₈)alkoxy” an alkoxy group whose hydrocarbon radical is as defined for the term “(C₁-C₈)alkyl”;

the term “(C₃-C₈)alkenoxy”, for example, the allyloxy, crotyloxy, 3-buten-1-yloxy, 1-penten-3-yloxy, 1-penten-4-yloxy or the 3-penten-2-yloxy group;

the term “(C₃-C₈)alkynyloxy”, for example, the propargyl, 1-butin-3-yloxy, 2-butin-1-yloxy or the 3-butin-1-yloxy group;

the term “(C₁-C₈)alkylthio” the alkylthio group whose alkyl radical is as defined for the term “(C₁-C₈)alkyl”;

the term “(C₁-C₈)alkylsulfinyl”, for example, methyl, ethyl, propyl, isopropyl, butyl, isobutyl, sec-butyl, tert-butylsulfinyl or the octylsulfinyl group and the term “(C₁-C₈)alkylsulfinyl”, for example, methyl, ethyl, propyl, isopropyl, butyl, isobutyl, sec-butyl, tert-butylsulfonyl or the octylsulfonyl group;

the term “(C₃-C₈)alkenylthio”, for example, allylthio, crotylthio, 3-buten-1-ylthio or the 3-penten-2-ylthio group;

the term “(C₃-C₈)alkynylthio”, for example, propargylthio, 1-butin-3-ylthio or the 3-butin-1-ylthio group;

the term “(C₁-C₈)alkylamino”, for example, methylamino, ethylamino, propylamino, isopropylamino, butylamino, isobutylamino, sec-butylamino, tert-butylamino, pentylamino, n-octylamino or the tert-octylamino group;

the term “(C₁-C₈)dialkylamino”, for example, dimethylamino, methylethylamino, diethylamino, dipropylamino or the dibutylamino group, but also cyclic systems such as the pyrrolidino or piperidino group and also those cyclic systems which contain a further heteroatom, such as, for example, the morpholino, thiomorpholino or piperazino group;

the term “trialkylsilyl” preferably the trimethylsilyl group;

the term “(C₁-C₈)alkanoyl”, for example, the formyl-, acetyl-, propionyl-, 2-methylpropionyl-, butyryl-, valeroyl-, pivaloyl-, hexanoyl-, heptanoyl- or octanoyl group;

the term “(C₁-C₈)alkoxycarbonyl”, for example, the methoxycarbonyl, ethoxycarbonyl, propoxycarbonyl, butoxycarbonyl, tert-butoxycarbonyl, pentyloxycarbonyl, hexyloxycarbonyl, or the octyloxycarbonyl group.

The substituents with which the various aliphatic, cycloaliphatic, aromatic and heterocyclic ring systems can be provided, for example, halogen, nitro, cyano, di-(C₁-C₄)alkylamino, (C₁-C₄)alkyl, (C₃-C₈)cycloalkyl, (C₁-C₄)trialkylsilyl, (C₁-C₄)alkoxy, (C₁-C₄)alkoxy-(C₁-C₄)alkyl, (C₁-C₂)alkoxy-[CH₂CH₂O]0,1,2-ethoxy, (C₁-C₄)alkylthio, (C₁-C₄)alkylsulfinyl, (C₁-C₄)alkylsulfonyl, phenyl, benzyl, phenoxy, halophenoxy, (C₁-C₄)alkylphenoxy, (C₁-C₄)alkoxyphenoxy, phenylthio, heterocyclyl, heterocyclylthio or heterocyclyloxy, it being possible for one or more, in the case of fluorine also up to the maximum number of, hydrogen atoms in the alkyl radicals and the radicals derived therefrom to be replaced by halogen, preferably chlorine or fluorine, where, in the event that these substituents are (C₁-C₄)alkyl, they may also be linked cyclically and where one or two aliphatic carbon units in these fused ring systems, such as, for example, the indane, di-, tetra- or decahydronaphthyl or benzocycloheptane system, may be replaced by heteroatom units such as oxygen or sulfur and where one or more, in the case of fluorine also up to the maximum number of, hydrogen atoms on the aliphatic carbon atom units can be replaced by halogen or (C₁-C₄)alkyl.

The definition that “a saturated carbon unit in the various R⁴ alkyl, cycloalkyl, alkenyl, alkynyl, cycloalkenyl radicals or the groups derived therefrom, such as the alkoxy, alkenyloxy, alkynyloxy, alkylthio, alkylsulfinyl, alkylsulfonyl, alkenylthio, alkynylthio, alkylamino, dialkylamino, trialkylsilyl, alkanoyl or alkoxycarbonyl group, can optionally be replaced by a carbonyl group or a heteroatom unit such as oxygen, S(O)ₓ where x=0, 1 or 2, or dimethylsilyl, and where additionally 3 to 8 atoms of these hydrocarbon radicals which are optionally modified as above can form a cycle and these hydrocarbon radicals, with or without the stated variations, are optionally substituted by one or more, preferably up to three, in the case of fluorine up to the maximum number of, identical or different radicals selected from the series consisting of halogen, aryl, aryloxy, arylthio, cycloalkoxy, cycloalkylthio, heterocyclyl, heterocyclyloxy, heterocyclylthio, alkanoyl, cycloalkanoyl, haloalkanoyl, aroyl, arylalkanoyl, cycloalkylalkanoyl, heterocyclylalkanoyl, alkoxycarbonyl, haloalkoxycarbonyl, cycloalkoxycarbonyl, cycloalkylalkoxycarbonyl, arylalkoxycarbonyl, heterocyclylalkoxycarbonyl, aryloxycarbonyl, heterocyclyloxycarbonyl, alkanoyloxy, haloalkanoyloxy, cycloalkanoyloxy, cycloalkylalkanoyloxy, aroyloxy, arylalkanoyloxy, heterocycloylalkanoyloxy, alkylsulfonyloxy, arylsulfonyloxy, hydroxyl, cyano, thiocyano or nitro,” is furthermore to be understood as meaning, for example,

alkoxyalkyl radicals such as, for example, the methoxymethyl, methoxyethyl or ethoxyethyl group; or

alkoxyalkoxyalkyl radicals such as, for example, the methoxy- or the ethoxyethoxyethyl group; or

alkylthioalkyl radicals such as, for example, the methyl- or the ethylthioethyl group; or

alkylsulfinylalkyl radicals such as, for example, the methyl- or ethylsulfinylethyl group; or

alkylsulfonylalkyl radicals such as, for example, the methyl or ethylsulfonylethyl group; or

alkyldimethylsilylalkyl radicals such as, for example, the trimethylsilylmethyl or the trimethylsilylethyl group; or

alkyldimethylsilyl radicals such as, for example, the trimethylsilyl, ethyldimethylsilyl, tert-butyldimethylsilyl or the octyidimethylsilyl group; or

cycloalkyldimethylsilyl radicals such as, for example, the cyclohexyldimethylsilyl group; or

aryidimethylsilyl radicals such as, for example, the phenyldimethylsilyl group; or

arylalkyldimethylsilyl radicals such as, for example, the benzyldimethylsilyl or the phenylethyldimethylsilyl group; or

alkanoylalkyl radicals such as, for example, the acetylmethyl or the pivaloylmethyl group; or cycloalkanoylalkyl radicals such as, for example, the cyclopropylcarbonylmethyl or the cyclohexylcarbonylmethyl group; or

haloalkanoylalkyl radicals such as, for example, the trifluoro- or trichloroacetylmethyl group; or

aroylalkyl radicals such as, for example, the benzoyl-, or naphthoylalkyl radicals such as, for example, the phenylacetylmethyl group; or

heterocyclylcarbonylalkyl radicals such as, for example, the thienyl- or pyridylacetylmethyl group; or

arylalkyl radicals such as, for example, the benzyl, the 2-phenylethyl, the 1-phenylethyl, the 1-methyl-1-phenylethyl group, the 3-phenylpropyl, the 4-phenylbutyl group, the 2-methyl-2-phenylethyl group or the 1-methyl- or 2-methylnaphthyl group; or

heterocyclylalkyl radicals such as, for example, the thienylmethyl, pyridylmethyl, furfuryl, tetrahydrofurfuryl, tetrahydropyranylmethyl or the 1,3-dioxolane-2-methyl group; or

aryloxyalkyl radicals such as, for example, the phenoxymethyl or naphthoxymethyl group; or

cycloalkyl radicals, monocyclic such as, for example, the cyclopropyl, cyclobutyl, cyclopentyl, cyclohexyl, cycloheptyl or cyclooctyl radical, bicyclic such as, for example, the norbornyl radical or the bicyclo[2,2,2]octyl radical, or fused, such as the decahydronaphthyl radical;

alkylcycloalkyl radicals such as, for example, the 4-methyl- or the 4-tert-butylcyclohexyl group or the 1-methylcyclopropyl, -cyclobutyl, -cyclopentyl or -cyclohexyl group;

cycloalkylalkyl radicals such as, for example, the cyclohexylmethyl or -ethyl group;

cycloalkylene radicals, monocyclic such as, for example, the cyclopentenyl, cyclohexenyl, cycloheptenyl or cyclooctenyl radical, bicyclic such as, for example, the norbornenyl or the bicyclo[2,2,2]octenyl radical, or fused, such as the various dihydro- or tetrahydronaphthyl radicals;

cycloalkylenealkyl radicals such as, for example, the 1-cyclohexenylmethyl or -ethyl radical;

or else haloalkyl derivatives of the corresponding groups, such as, for example, haloalkyl, haloalkoxyalkyl, alkoxyhaloalkyl, haloalkylcycloalkyl or halocycloalkyl radicals.

Furthermore, the term that “two radicals R⁴ which are bonded to adjacent carbon atoms (D, E; L, D or G, L) together with these carbon atoms form an unsaturated 5- or 6-membered carbocyclic ring which, if it takes the form of a 5-membered ring, may contain an oxygen or sulfur atom in place of CH₂ or, if it takes the form of a 6-membered ring, may contain one or two nitrogen atoms in place of one or two CH units, and which is optionally substituted by 1, 2 or 3 identical or different radicals, these radicals denoting (C₁-C₄)alkyl, (C₁-C₄)haloalkyl, (C₁-C₄)alkoxy or hydroxyl, preferably methyl, trifluoromethyl, halogen, preferably fluorine or chlorine, or methoxy, where, in the event that it takes the form of a nitrogen heterocycle and the substituent(s) take(s) the form of hydroxyl groups, the ring system may also exist in the tautomeric keto form, in which case a (C₁-C₄)alkyl substituent which may be present may also be attached to a nitrogen atom, or

two radicals R⁴ together with the carbon atoms (GL, LD, DE) to which they are bonded form a saturated 5-, 6- or 7-membered carbocyclic ring which is optionally substituted by 1, 2 or 3 (C₁-C₄)alkyl groups”, for the radical Az of the formula (II), are to be understood as meaning, for example, benzo-fused azoles such as benzimidazole or benzotriazole or pyridoazoles such as 1H-imidazo-[4,5-b]pyridine or cycloalkenoazoles, such as 4,5,6,7-tetrahydroindazole, 4,5,6,7-tetrahydro-1H-benzo[d]-imidazole, 2,4,5,6-tetrahydrocyclopentapyrazole or 4,5,6,7-tetrahydro-2H-indazole, or other fused systems, such as purine or theophyllin.

What has been said above applies analogously to homologs or their derived radicals.

The present invention relates to the compounds of the formula (I) in the form of the free base or of an acid addition salt. Acids which can be used for salt formation are, e.g., inorganic acids such as hydrochloric acid, hydrobromic acid, nitric acid, sulfuric acid, phosphoric acid, or organic acids such as formic acid, acetic acid, propionic acid, malonic acid, oxalic acid, fumaric acid, adipic acid, stearic acid, oleic acid, methanesulfonic acid, benzenesulfonic acid or toluenesulfonic acid.

Some of the compounds of the formula (I) have one or more asymmetric carbon atoms or stereoisomers on double bonds. Enantiomers or diastereomers are therefore possible. The invention encompasses not only the pure isomers, but also their mixtures. The diastereomer mixtures can be resolved into the components by customary methods, for example by selective crystallization from suitable solvents or by chromatography. Racemates can be resolved by customary methods to give the enantiomers, for example by salt formation with a chiral enantiomerically pure acid, separation of the diastereomeric salts and setting free the pure enantiomers by means of a base.

The present invention also relates to processes for the preparation of compounds of the formula (I).

For example, a process for the preparation of compounds of the formula (I) where

a) A, R¹, X, n and Az are as defined for formula (I) and A′ is nitrogen comprises A. reacting a compound of the formula (III)

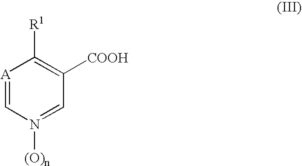

in which A, R¹ and n have the abovementioned meanings, if appropriate in the form of an activated derivative of this acid, in the presence of a base with a compound of

the formula (IV)

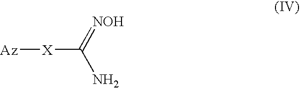

in which X and Az are as defined above.

An example of an activated derivative which can be employed is an acyl halide, an ester or an anhydride. Bases which are suitable are, e.g., amines such as, for example, triethylamine, diisopropylethylamine, pyridine or lutidine, alkali metal hydroxides, alkali metal alkoxides such as sodium methoxide or potassium tert-butoxide, or alkyl metal compounds such as butyllithium.

The reaction described can be carried out as a one-step process or as a two-step process, depending on the choice of the conditions, given, as intermediates, compounds of the formula (V)

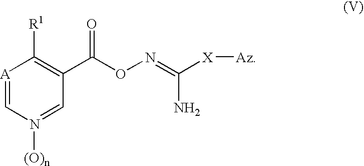

in which A, R¹, n, X and Az are as defined above.

Compounds of the formula (V) can be cyclized to give the 1,2,4-oxadiazoles, for example, by heating in an inert solvent at temperatures of up to 180° C. Compounds of the formula (V) can also be obtained directly from the acid of the formula (III) and amidoximes of the formula (IV) by using a dehydrating reagent such as dicyclohexylcarbodiimide, 1-ethyl-3-(3-dimethylaminopropyl)carbodiimide or N,N′-carbonyldiimidazole.

Both acids of the formula (III) and amidoximes of the formula (IV) are known or can be prepared by processes known from the literature (see, for example, Houben-Weyl, Methoden der Organischen Chemie [Methods in Organic Chemistry], Volume X/4, pages 209-212; EP-A 0 580 374; G. F. Holland, J. N.Pereira, J. Med. Chem. 1967, 10, 149).

Compounds of the formula (I) in which the radicals are defined as above under a) can furthermore be prepared by a process in which

B. a compound of the formula (VI)

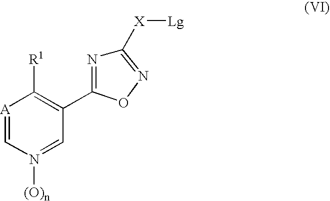

in which A, R¹, n and X are as defined above for formula (I) and Lg is a leaving group such as, for example, halogen, alkanesulfonyloxy, arylsulfonyloxy, alkylsulfonyl or arylsulfonyl,

is reacted with an optionally substituted azole of the formula (VII)

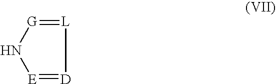

in which D, E, G, L are as defined above for formula (II).

The above-described substitution reaction is known in principle. The leaving group Lg can be varied within wide limits and can be, for example, a halogen atom such as fluorine, chlorine, bromine or iodine, or alkanesulfonyloxy such as methane-, trifluoromethane- or ethanesulfonyloxy, or arylsulfonyloxy, such as benzenesulfonyloxy or toluenesulfonyloxy, or alkylsulfonyl, such as methyl- or ethylsulfonyl, or arylsulfonyl, such as phenyl- or toluenesulfonyl.

In general, the abovementioned reaction is carried out in a temperature range of from 20 to 150° C., expediently in the presence of a base and if appropriate in an inert organic solvent such as N,N-dimethylformamide, N,N-dimethylacetamide, dimethylsulfoxide, N-methylpyrrolidin-2-one, dioxane, tetrahydrofuran, 4-methyl-2-pentanone, methanol, ethanol, butanol, ethylene glycol, ethylene glycol dimethyl ether, toluene, chlorobenzene or xylene. Mixtures of these may also be used.

Examples of suitable bases are the carbonates, hydrogen carbonates, hydroxides, amides or hydrides of alkali metals or alkaline earth metals, such as sodium carbonate, sodium hydrogen carbonate, potassium carbonate, sodium hydroxide, sodium amide or sodium hydride, or organic bases such as triethylamine or pyridine. A second equivalent of the azole (VII) may also be employed as auxiliary base.

To prepare compounds of the formula (I) in which

b) A, R¹, R², X, n and Az are as defined for formula (I) and A′ is CH, a procedure is expediently followed which is analogous to the substitution reaction described above for B. The corresponding starting materials have been described (DE-198 58 192) or can be prepared analogous to known processes.

Collections of compounds of the formula (I) which can be synthesized by the abovementioned schemes may also be prepared in parallel, and this may be effected manually or in a semiautomated or fully automated fashion. For example, it is possible to automate the procedure of the reaction, work-up or purification of the products or intermediates. In total, this is to be understood as a procedure as is described, for example, by S. H. DeWitt in “Annual Reports in Combinatorial Chemistry and Molecular Diversity: Automated synthesis”, Volume 1, Verlag Escom 1997, pages 69 to 77.

A series of commercially available apparatuses may be used for the parallel procedure of the reaction and work-up as are offered, for example, by Stem Corporation, Woodrolfe Road, Tollesbury, Essex, England, H+P Labortechnik GmbH, Bruckmannring 28, 85764 Oberschleiβheim, Germany, or Radleys, Shirehill, Saffron Walden, Essex, CB-II:3AZ, England. Equipment which is available for the parallel purification of compounds of the formula (I) or of intermediates obtained during the preparation are, inter alia, chromatography apparatuses, for example those of ISCO, Inc., 4700 Superior Street, Lincoln, Nebr. 68504, USA.

The apparatuses mentioned result in a modular procedure, in which the individual passes are automated, but manual operations must be carried out between the passes. This can be circumvented by employing partially or fully integrated automation systems in which the automation modules in question are operated by, for example, robots. Such automation systems can be obtained, for example, from Zymark Corporation, Zymark Center, Hopkinton, Mass. 01748, USA.

In addition to the methods described here, compounds of the formula (I) can be prepared completely or partially by solid-phase-aided methods. To this end, individual intermediates or all intermediates of the synthesis or of a synthesis adapted to suit the procedure in question are bound to a synthetic resin. Solid-phase-aided synthetic methods are described extensively in the specialist literature, for example Barry A. Bunin in “The Combinatorial Index”, Verlag Academic Press, 1998.

The use of solid-phase-aided synthetic methods allows a series of protocols known from the literature, which, in turn, can be carried out manually or in an automated fashion. For example, the tea-bag method (Houghten, U.S. Pat. No. 4,631,211; Houghten et al., Proc. Natl. Acad. Sci, 1985, 82, 5131-5135) can be partially automated using products by IRORI, 11149 North Torrey Pines Road, La Jolla, Calif. 92037, USA. Solid-phase-aided parallel syntheses are successfully automated by, for example, apparatuses by Argonaut Technologies, Inc., 887 Industrial Road, San Carlos, Calif. 94070, USA or MultiSynTech GmbH, Wullener Feld 4, 58454 Witten, Germany.

The preparation in accordance with the processes described herein yields compounds of the formula (I) in the form of substance collections, which are termed libraries. The present invention also relates to libraries comprising at least two compounds of the formula (I).

The active substances of the formula (I) are suitable for controlling animal pests, in particular insects, arachnids, helminths and molluscs, very particularly preferably for controlling insects and arachnids found in agriculture, in livestock production, in forests, in the protection of stored products and materials, and in the hygiene sector, while being well tolerated by plants and having a favorable toxicity to warm-blooded species. They are active against normally-sensisitve and resistant species and against all or individual developmental stages. The abovementioned pests include: From the order of the Acarina, for example, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp., Eutetranychus spp.

From the order of the Isopoda, for example, Oniscus aselus, Armadium vulgare, Porcellio scaber.

From the order of the Diplopoda, for example, Blaniulus guttulatus.

From the order of the Chilopoda, for example, Geophilus carpophagus, Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blafta orientalis, Periplaneta americana, Leucophaea maderae, Blattella germanica, Acheta domesticus, Gryllotalpa spp., Locusta migratoria migratorioides, Melanoplus differentialis, Schistocerca gregaria.

From the order of the Isoptera, for example, Reticulitermes spp.

From the order of the Anoplura, for example, Phylloxera vastatrix, Pemphigus spp., Pediculus humanus corporis, Haematopinus spp., Linognathus spp.

From the order of the Mallophaga, for example, Trichodectes pp., Damalinea spp.

From the order of the Thysanoptera, for example, Hercinothrips femoralis, Thrips tabaci.

From the order of the Heteroptera, for example, Eurygaster spp., Dysdercus intermedius, Piesma quadrata, Cimex lectularius, Rhodnius prolixus, Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Empoasca spp., Euscelus bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Cacoecia podana, Capua reticulana, Choristoneura fumiferana, Clysia ambiguella, Homona magnanima, Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylloides chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrynchus assimilis, Hypera postica, Dermestes spp., Trogoderma, Anthrenus spp., Attagenus spp., Lyctus spp., Meligethes aeneus, Ptinus spp., Niptus hololeucus, Gibbium psylloides, tribolium spp., Tenebrio molitor, Agriotes spp., Conoderus spp., Melolontha melolontha, Amphimallon solstitialis, Costelytra zealandica.

From the order of the Hymenoptera, for example, Diprion spp., Hoplocampa spp., Lasius spp., Monomorium pharaonis, Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hypobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa.

From the order of the Siphonaptera, for example, Xenopsylla cheopsis, Ceratophyllus spp.

From the order of the Arachnida, for example, Scorpio maurus, Latrodectus mactans.

From the class of the helminths, for example, Haemonchus, Trichostrongulus, Ostertagia, Cooperia, Chabertia, Strongyloides, Oesophagostomum, Hyostrongulus, Ancylostoma, Ascaris, Heterakis and Fasciola.

From the class of the Gastropoda, for example, Deroceras spp., Arion spp., Lymnaea spp., Galba spp., Succinea spp., Biomphalaria spp., Bulinus spp., Oncomelania spp.

From the class of the Bivalva, for example, Dreissena spp.

The invention also relates to compositions, in particular to insecticidal and acaricidal compositions, which comprise the compounds of the formula (I) in addition to one or more suitable formulation auxiliaries.

In general, the active substances of the formula (I) amount to 1 to 95% by weight of the compositions according to the invention.

The latter can be formulated in various ways, depending on the biological and/or chemico-physical parameters which prevail. The following are preferred possibilities of formulation:

Wettable powders (WP), emulsifiable concentrates (EC), aqueous solutions (SL), emulsions, sprayable solutions, oil- or water-based dispersions (SC), suspoemulsions (SE), dusts (DP), seed-dressing materials, granules in the form of microgranules, spray granules, coated granules and adsorption granules, water-dispersible granules (WG), ULV formulations, microcapsules, waxes or baits.

These individual formulation types are known in principle and described, for example, in:

Winnacker-Küchler, “Chemische Technologie” [Chemical Engineering], Volume 7, C. Hauser Verlag Munich, 4th Edition 1986; van Falkenberg, “Pesticides Formulations”, Marcel Dekker N.Y., 2nd Ed. 1972-73; K. Martens, “Spray Drying Handbook”, 3rd Ed. 1979, G. Goodwin Ltd. London.

The formulation auxiliaries required, such as inert materials, surfactants, solvents and further additives, are also known and described, for example, in:

Watkins, “Handbook of Insecticide Dust Diluents and Carriers”, 2nd Ed., Darland Books, Caldwell N.J.; H. v. Olphen, “Introduction to Clay Colloid Chemistry”, 2nd Ed., J. Wiley & Sons, N.Y.; Marsden, “Solvents Guide”, 2nd Ed., Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1967; Winnacker-Küchler, “Chemische Technologie”, Volume 7, C. Hanser Verlag Munich, 4th Edition 1986.

Based on these formulations, it is also possible to prepare combinations with other pesticidally active substances, fertilizers and/or growth regulators, for example in the form of a readymix or a tank mix. Wettable powders are preparations which are uniformly dispersible in water and which, besides the active substance, also comprise, in addition to a diluent or inert material, wetters, for example polyoxethylated alkylphenols, polyoxethylated fatty alcohols, alkylsulfonates or alkylphenolsulfonates, and dispersants, for example, sodium lignosulfonate, sodium 2,2′-dinaphthylmethane-6,6′-disulfonate.

Emulsifiable concentrates are prepared by dissolving the active substance in an organic solvent, for example butanol, cyclohexanone, dimethylformamide, xylene or else higher-boiling aromatics or hydrocarbons with addition of one or more emulsifiers. Emulsifiers which can be used are, for example: calcium salts of alkylarylsulfonic acids, such as calcium dodecylbenzenesulfonate, or nonionic emulsifiers, such as fatty acid polyglycol esters, alkylaryl polyglycol ethers, fatty alcohol polyglycol ethers, propylene oxide/ethylene oxide condensates, alkyl polyethers, sorbitan fatty acid esters, polyoxyethylene sorbitan fatty acid esters or polyoxethylene sorbitol esters.

Dusts are obtained by grinding the active substance with finely divided solid materials, for example talc or natural clays, such as kaolin, bentonite or pyrophyllite, or diatomaceous earth. Granules can be produced either by spraying the active substance onto adsorptive, granulated inert material or by applying active substance concentrates onto the surface of carriers such as sand, kaolinites or of granulated inert material, by means of binders, for example polyvinyl alcohol, sodium polyacrylate or alternatively mineral oils. Suitable active substances can also be granulated in the manner which is conventional for the production of fertilizer granules, if desired in a mixture with fertilizers.

The active substance concentration in wettable powders is, for example, about 10 to 90% by weight; the remaining 100% by weight is composed of conventional formulation components. In the case of emulsifiable concentrates, the active substance concentration can be approximately 5 to 80% by weight. Formulations in the form of dusts usually comprise 5 to 20% by weight of active substance, sprayable solutions approximately 2 to 20% by weight. In the case of granules, the active substance content depends partly on whether the active compound is in liquid or solid form and on which granulation auxiliaries, fillers etc. are being used.

In addition, the active substance formulations mentioned comprise, if appropriate, the adhesives, wetters, dispersants, emulsifiers, penetrants, solvents, fillers or carriers which are conventional in each case.

For use, the concentrates which are present in commercially available form are diluted, if appropriate, in the customary manner, for example using water in the case of wettable powders, emulsifiable concentrates, dispersions and in some cases also microgranules. Preparations in the form of dusts and granulated preparations and sprayable solutions are conventionally not further diluted with other inert materials prior to use.

The application rate required varies with the external conditions such as temperature, humidity and the like. It can vary within wide limits, for example between 0.0005 and 10.0 kg/ha or more of active substance, but it is preferably between 0.001 and 5 kg/ha.

The active substances according to the invention may exist, in their commercially available formulations and in the use forms prepared with these formulations, either alone or as mixtures with other active substances such as insecticides, attractants, sterilants, acaricides, nematicides, fungicides, growth regulators or herbicides.

The pesticides with which compounds of the formula (I) can be combined include, for example, phosphoric esters, carbamates, carboxylates, formamidines, tin compounds and substances produced by microorganisms.

Preferred components in mixtures are

1. from the group of the phosphorus compounds

acephate, azamethiphos, azinphos-ethyl, azinphos-methyl, bromophos, bromophos-ethyl, cadusafos (F-67825), chlorethoxyphos, chlorfenvinphos, chlormephos, chlorpyrifos, chlorpyrifos-methyl, demeton, demeton-S-methyl, demeton-S-methyl sulfone, dialifos, diazinon, dichlorvos, dicrotophos, dimethoate, disulfoton, EPN, ethion, ethoprophos, etrimfos, famphur, fenamiphos, fenitriothion, fensulfothion, fenthion, fonofos, formothion, fosthiazate (ASC-66824) heptenophos, isazophos, isothioate, isoxathion, malathion, methacrifos, methamidophos, methidathion, salithion, mevinphos, monocrotophos, naled, omethoate, oxydemeton-methyl, parathion, parathion-methyl, phenthoate, phorate, phosalone, phosfolan, phosphocarb (BAS-301), phosmet, phosphamidon, phoxim, pirimiphos, primiphos-ethyl, pirimiphos-methyl, profenofos, propaphos, proetamphos, prothiofos, pyraclofos, pyridapenthion, quinalphos, sulprbfos, temephos, terbufos, tebupirimfos, tetrachlorvinphos, thiometon, triazophos, trichlorphon, vamidothion;

2. from the group of the carbamates

alanycarb (OK-135), aldicarb, 2-sec-butyl phenylmethyl carbamate (BPMC), carbaryl, carbofuran, carbosulfan, cloethocarb, benfuracarb, ethiofencarb, furathiocarb, HCN-801, isoprocarb, methomyl, 5-methyl m-cumenylbutyryl(methyl)carbamate, oxamyl, pirimicarb, propoxur, thiodicarb, thiofanox, 1-methylthio(ethylideneamino)-N-methyl-N-(morpholinothio)carbamate (UC 51717), triazamate;

3. from the group of the carboxylic esters

acrinathrin, allethrin, alphametrin, 5-benzyl-3-furylmethyl (E)-, (1R)-cis-2,2-di-methyl-3-(2-oxothiolan-3-ylidenemethyl) cyclopropanecarboxylate, beta-cyfluthrin, beta-cypermethrin, bioallethrin, bioallethrin ((S)cyclopentyl isomer), bioresmethrin, bifenthrin, (RS)-1-cyano-1-(6-phenoxy-2-pyridyl)methyl (IRS)-trans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flucythrinate, flumethrin, fluvalinate (D isomer), imiprothrin (S41311), lambda-cyhalothrin, permethrin, phenothrin ((R) isomer), prallethrin, pyrethrins (natural products), resmethrin, tefluthrin, tetramethrin, theta-cypermethrin (TD-2344), tralomethrin, transfluthrin, zeta-cypermethrin (F-56701);

4. from the group of the amidines

amitraz, chlorodimeform;

5. from the group of the tin compounds

cyhexatin, fenbutatin oxide;

6. others

abamectin, ABG-9008, acetamiprid, Anagrapha falcitera, AKD-1022, AKD-3059, ANS-118, Bacillus thuringiensis, Beauveria bassiana, bensultap, bifenazate (D-2341), binapacryl, BJL-932, bromopropylate, BTG-504, BTG-505, buprofezin, camphechlor, cartap, chlorbenzilate, chlorfenapyr, chlorfluazuron, 2-(4-chlorophenyl)-4,5-diphenylthiophene (UBI-T 930), chlorfentezine, chromafenozide (ANS-118), CG-216, CG-217, CG-234, A-184699, (2-naphthylmethyl)cyclopropanecarboxylate (Ro12-0470), cyromazin, diacloden (thiamethoxam), diafenthiuron, ethyl N-(3,5-dichloro4-(1,1,2,3,3,3-hexafluoro-1-propyloxy)phenyl)carbamoyl)-2-chlorobenzocarboximidate, DDT, dicofol, diflubenzuron, N-(2,3-dihydro-3-methyl-1,3-thiazol-2-ylidene)-2,4-xylidine, dinobuton, dinocap, diofenolan, DPX-062, emamectin benzoate (MK-244), endosulfan, ethiprole (sulfethiprole), ethofenprox, etoxazole (YI-5301), fenazaquin, fenoxycarb, fipronil, fluazuron, flumite (Flufenzine, SZI-121), 2-fluoro-5-(4-(4-ethoxyphenyl)-4-methyl-1-pentyl)diphenyl ether (MTI 800), granulosis and nuclear polyhedrosis viruses, fenpyroximate, fenthiocarb, flubenzimine, flucycloxuron, flufenoxuron, flufenprox (ICI-A5683), fluproxyfen, gamma-HCH, halofenozide (RH-0345), halofenprox (MTI-732), hexaflumuron (DE_{—}473), hexythiazox, HOI-9004, hydramethylnon (AC 217300), lufenuron, imidacloprid, indoxacarb (DPX-MP062), kanemite (AKD-2023), M-020, MTI-446, ivermectin, M-020, methoxyfenozide (intrepid, RH-2485), milbemectin, NC-196, neemgard, nitenpyram (TI-304), 2-nitromethyl-4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), pyriproxyfen (S-71639), NC-196, NC-111, NNI-9768, novaluron (MCW-275), OK-9701, OK-9601, OK-9602, propargite, pymethrozine, pyridaben, pyrimidifen (SU-8801), RH-0345, RH-2485, RYI-210, S-1283, S-1833, SB7242, SI-8601, silafluofen, silomadine (CG-177), spinosad, SU-9118, tebufenozide, tebufenpyrad (MK-239), teflubenzuron, tetradifon, tetrasul, thiacloprid, thiocyclam, TI-1435, tolfenpyrad (OMI-88), triazamate (RH-7988), triflumuron, verbutin, vertalec (Mykotal), YI-5301.

The abovementioned components constitute known active substances, many of which are described in C. D. S. Tomlin, S. B. Walker, The Pesticide Manual, 11th Edition (1997), British Crop Protection Council.

The active substance content of the use forms prepared from the commercially available formulations can vary within wide limits, the active substance concentration of the use forms can be from 0.0001 up to 95% by weight of active substance, preferably between 0.0001 and 1% by weight. They are applied in a customary manner adapted to suit the use forms.

Preferred is the use in economically important crops of useful plants and ornamentals, for example of cereals, such as wheat, barley, rye, oats, millet and sorghum, rice, cassaya and maize, or else crops of sugarbeet, cotton, soya, oilseed rape, potatoes, tomatoes, peas and other vegetables, or in orchards.

Besides the applications methods mentioned so far, the active substances of the formula (I) according to the invention show an outstanding systemic action. The active substances can therefore also be introduced into the plants via subterranean or aerial plant organs (roots, stalks, leaves) when the active substances are applied to the immediate environment of the plants either in liquid or in solid form (for example granules in the case of soil application, application into paddy fields).

In addition, the active substances according the invention can be employed in a particular manner for the treatment of vegetative and generative propagation materials, such as seed of, for example, cereals, vegetables, cotton, rice, sugarbeet and other crop plants and ornamentals, of bulbs, cuttings and tubers of other crop plants and ornamentals which are propagated by vegetative propagation. To this end, treatment may take place prior to sowing or the planting procedure (for example by specific seed-dressing techniques, by dressing in liquid or solid form, or by the seed box treatment), during the sowing procedure or during planting, or after the sowing or planting procedure, using specific application techniques (for example row treatment). The active substance quantity applied may vary within a substantial range, depending on the purpose. In general, the application rates are between 1 g and 10 kg of active substance per hectare of soil surface.

The active substances according to the invention are also suitable for application in the field of veterinary medicine, preferably for controlling ectoparasites, and in the field of animal keeping.

Here, the active substances according to the invention are preferably applied in a known manner, such as by oral administration in the form of, for example, tablets, capsules, drinks, granules, by dermal application in the form of, for example, dipping, spraying, pouring-on and spotting-on and dusting, and by parenteral administration, for example in the form of an injection.

Accordingly, the compounds of the formula (I) according to the invention can also be employed particularly advantageously in livestock keeping (for example cattle, sheep, pigs and poultry such as chickens and geese). In a preferred embodiment of the invention, the compounds of the formula (I) are administered to the animals orally, if appropriate in suitable fromulations (cf. above) and if appropriate together with the drinking water or the feed. Since excretion with the feces is highly efficient, this allows the development of insects in the animals' feces to be prevented in a very simple fashion. The dosages and formulations which are suitable in each case depend in particular on the species and developmental stage of the livestock and also on the risk of infection and can readily be determined and specified by the customary methods. For example, the compounds can be employed in cattle in dosages of 0.01 to 1 mg/kg bodyweight.

The active compound content of the use forms prepared from the commercially available formulations intended for use in veterinary medicine can vary within wide ranges; the active substance concentration of the use forms can be from 0.0001 up to 95% by weight of active substance, preferably between 0.0001 and 1% by weight. They are used in a customary manner adapted to suit the use forms. The invention therefore also relates to the use of compounds of the formula (I) for the preparation of an animal medicine, preferably for controlling ectoparasites.

The compounds of the formula (I) can also be employed for controlling harmful organisms in crops of known genetically modified plants, or genetically modified plants yet to be developed. As a rule, the transgenic plants are distinguished by particular, advantageous properties, for example by resistances to certain crop protection agents, resistances to plant diseases or to pathogens of plant diseases such as certain insects or microorganisms such as fungi, bacteria or viruses. Other particular properties relate to for example the harvested material with regard to quantity, quality, shelf-life, composition and specific constituents. For example, transgenic plants with an increased starch content or with an altered starch quality or those whose harvested material has a different fatty acid spectrum are known.

Preferred is the use in economically important transgenic crops of useful plants and ornamentals, for example of cereals such as wheat, barley, rye, oats, sorghum and millet, rice, cassaya and maize, or else crops of sugarbeet, cotton, soya, oilseed rape, potatoes, tomatoes, peas and other vegetables.

When used in transgenic cultures, in particular those with resistances to insects, effects are frequently observed—in addition to the effects against harmful organisms which can be observed in other crops—which are specific for application in the particular transgenic crop, for example an altered, or specifically widened, spectrum of pests which can be controlled, or altered application rates which can be employed for application.

The invention therefore also relates to the use of compounds of the formula (I) for controlling harmful organisms in transgenic crop plants.

In addition to the direct application to the pests, application of the compound according to the invention comprises any other application where compounds of the formula (I) act on the pests. Such indirect applications can be, for example, the use of compounds which break down, or are broken down, to give compounds of the formula (I), for example in the soil, the plants or the pests.

In addition to their lethal effect on pests, the compounds of the formula (I) are also distinguished by a pronounced repellant effect.

A repellant for the purposes of the invention is a substance or substance mixture which has a warding-off or fending-off effect on other live organisms, in particular harmful pests and nuisance pests. The term also encompasses effects such as the antifeeding effect, where the intake of feed is disturbed or prevented (antifeedant effect), supression of oviposition, or an effect on the development of the population.

The invention therefore also relates to the use of compounds of the formula (I) for achieving the abovementioned effects, in particular in the case of the pests stated in the biological examples.

The invention also relates to a method of repelling harmful organisms, where one or more compounds of the formula (I) are applied to the site from which the harmful organisms are to be fended off or warded off.

In the case of a plant, application may mean, for example, a treatment of the plant, but also of the seed.

As regards the effect on populations, it is interesting to note that effects can also be observed in succession during the development of a population, where summation may take place. In such a case, the individual effect itself may only have an efficacy of markedly less than 100% but in total an efficacy of 100% is still achieved in the end.

Moreover, the compounds of the formula (I) are distinguished by the fact that the composition is usually applied earlier than in the case of a direct control, if the abovementioned effects are to be exploited. The effect frequently lasts over a long period, so that a duration of action of over 2 months is achieved.

The effects are not only found in insects, but also in spider mites and molluscs.

The content of the German Patent Application 19962901.3, whose priority the present application claims, and the appended abstract is herewith expressly referred to; it is incorporated herein by reference.

The examples which follow are intended to illustrate the invention without limiting it thereto.

### A. SYNTHESIS EXAMPLES

### Example 1

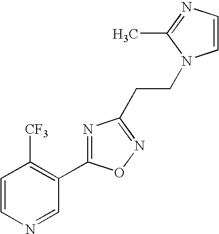

2.90 g (8.5 mmol) of 3-(2-methyl-imidazol-1-yl) O-(4-trifluoromethylnicotinoyl)propionamidoxime and 0.95 g (8.5 mmol) of potassium tert-butoxide were stirred for 4 hours at 50-60° C. in 100 ml of tetrahydrofuran. After concentration, the residue was taken up in ethyl acetate/water, and the organic phase was dried and concentrated. For purification, the product was chromatographed on silica gel (ethyl acetate/methanol 7:3). This gave 1.1 g (40.0% of theory) of product as colorless oil.

¹H NMR (CDCl₃): 9.30 (S, 1H), 9.05 (d, 1H), 7.80 (d, 1H) pyridine H; 6.88, 6.92 (2S, 2H, imidazole H); 4.39 (tr, 2H, CH₂), 3.30 (tr, 2H, CH₂), 2.40 (s, 3H, CH₃).

Preparation of the Starting Material 3-(2-methylimidazol-1-yl) O-(4-trifluoromethylnicotinoyl)propionamidoxime

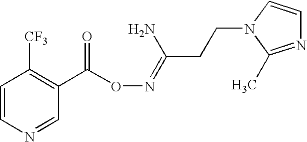

3.82 g (20 mmol) of 4-trifluoromethylnicotinic acid and 3.24 g (20 mmol) of carbonyldiimidazole were stirred in 100 ml of dry tetrahydrofuran at 50° C. until the evolution of CO₂ had ceased. The mixture was allowed to cool, and a solution of 4.21 g (25 mmol) of 3-(2-methyl-imidazol-1-yl)propionamidoxime in 50 ml of tetrahydrofuran was added. Stirring was continued for 6 h at 50° C. The mixture was concentrated and the product taken up in water/dichloromethane. For purification, the mixture was chromatographed on silica gel using ethyl acetate. This gave 3.5 g (51.3% of theory) of product as colorless solid. M.p. 137-138° C.

Preparation of 3-(2-methyl-imidazol-1-yl)propionamidoxime

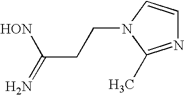

31.97 g of 30% methanolic sodium methoxide solution were added to 20 g (0.148 mol) of 3-(2-methyl-imidazol-1-yl)propionitrile and 12.4 g (0.178 mol) of hydroxylammonium chloride, and the mixture was stirred for 8 h at 50° C. After cooling, the sodium chloride was filtered off and the filtrate was concentrated. This gave 18.3 g (73.5% of theory) of product as colorless oil which was employed in the next step without further purification.

### Example 2

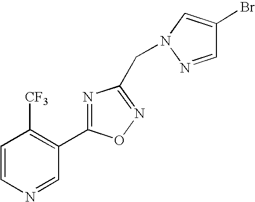

670 mg (4.6 mmol) of 4-bromopyrazole was added to a suspension of 40 mg (1.4 mmol) of sodium hydride (80% dispersion in oil) in 10 ml of dimethylformamide, and the mixture was stirred for 30 minutes at 40° C. 300 mg (1.1 mmol) of 3-chloromethyl-5-(4-trifluoromethyl-3-pyridyl)-1,2,4-oxadiazole (Ex. 373 in WO-A 98/57969), dissolved in a little dimethylformamide, were added, and stirring was continued for 6 h at room temperature. For work-up, the mixture was diluted with toluene, washed with water, and the organic phase was dried and concentrated. For purification, the product was chromatographed on silica gel using heptane/ethyl acetate 2:1. This gave 210 mg of product (49% of theory) as colorless oil.

The compounds listed in the tables which follow are prepared analogously.

TABLE 1

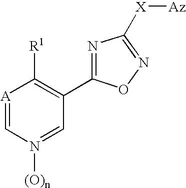

| Ex. | | | | | | |
|---|---|---|---|---|---|---|
| No. | A | n | R¹ | X | Az | m.p. [° C.] |
| 1 | CH | 0 | CF₃ | —CH₂ | 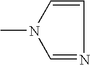 | 90-92 |
| 2 | N | 0 | CF₃ | ″ | ″ | |
| 3 | CH | 1 | CF₃ | ″ | ″ | |
| 4 | CH | 0 | CF₂ | ″ | ″ | |
| | | | Cl | | | |
| 5 | CH | 0 | CF₃ | CH₂CH₂ | ″ | oil |
| 6 | N | 0 | CF₃ | CH₂CH₂ | ″ | |
| 7 | CH | 0 | CF₃ | (CH₂)₃ | ″ | |
| 8 | CH | 0 | CF₃ | (CH₂CH(CH₃) | ″ | |
| 9 | CH | 0 | CF₃ | (CH₂)₄ | ″ | |
| 10 | CH | 0 | CF₃ | (CH₂)₅ | ″ | |
| 11 | CH | 0 | CF₃ | (CH₂)₈ | ″ | |
| 12 | CH | 0 | CF₃ | CH₂ | 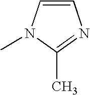 | |
| 13 | CH | 0 | CF₃ | (CH₂)₂ | ″ | oil |
| 14 | CH | 0 | CF₃ | (CH₂)₃ | ″ | |
| 15 | CH | 0 | CF₃ | CH₂ | 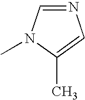 | 72-73 |
| 16 | CH | 0 | CF₃ | (CH₂)₂ | ″ | |
| 17 | CH | 0 | CF₃ | (CH₂)₃ | 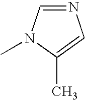 | |
| 18 | CH | 0 | CF₃ | CH₂ | 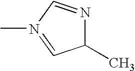 | 64-65 |
| 19 | CH | 0 | CF₃ | (CH₂)₂ | ″ | |
| 20 | CH | 0 | CF₃ | (CH₂)₃ | ″ | |
| 21 | CH | 0 | CF₃ | CH₂ | 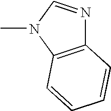 | |
| 22 | CH | 0 | CF₃ | (CH₂)₂ | ″ | 138-139 |
| 23 | CH | 0 | CF₃ | (CH₂)₃ | ″ | |
| 24 | CH | 0 | CF₃ | CH₂ | 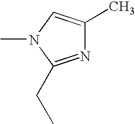 | |
| 25 | CH | 0 | CF₃ | (CH₂)₂ | ″ | 72-74 |
| 26 | CH | 0 | CF₃ | (CH₂)₃ | ″ | |
| 27 | CH | 0 | CF₃ | CH₂ | 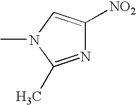 | 114-117 |
| 28 | CH | 0 | CF₃ | (CH₂)₂ | ″ | |
| 29 | CH | 0 | CF₃ | (CH₂)₃ | ″ | |
| 30 | CH | 0 | CF₃ | CH₂ | pyrazole | oil |
| 31 | N | 0 | CF₃ | CH₂ | ″ | |
| 32 | CH | 1 | CF₃ | CH₂ | ″ | |
| 33 | CH | 0 | CF₂ | CH₂ | ″ | |
| | | | Cl | | | |
| 34 | CH | 0 | CF₃ | CH₂CH₂ | pyrazole | oil |
| 35 | CH | 0 | CF₃ | (CH₂)₃ | ″ | |
| 36 | CH | 0 | CF₃ | (CH₂)₄ | ″ | |
| 37 | CH | 0 | CF₃ | (CH₂)₅ | ″ | |
| 38 | CH | 0 | CF₃ | (CH₂)₆ | ″ | |
| 39 | CH | 1 | CF₃ | (CH₂)₂ | ″ | |
| 40 | CH | 0 | CF₃ | CH₂CH(CH₃) | ″ | |
| 41 | CH | 0 | CF₃ | CH₂ | 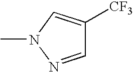 | oil |
| 42 | CH | 0 | CF₃ | (CH₂)₂ | ″ | |
| 43 | CH | 0 | CF₃ | (CH₂)₃ | ″ | |
| 44 | CH | 0 | CF₃ | CH₂ | 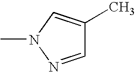 | oil |
| 45 | CH | 0 | CF₃ | (CH₂)₂ | ″ | oil |
| 46 | CH | 0 | CF₃ | (CH₂)₃ | ″ | |
| 47 | CH | 0 | CF₃ | CH₂ | 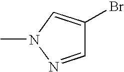 | oil |
| 48 | CH | 0 | CF₃ | (CH₂)₂ | ″ | |
| 49 | CH | 0 | CF₃ | (CH₂)₃ | ″ | |
| 50 | CH | 0 | CF₃ | CH₂ | 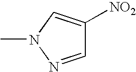 | 129-130 |
| 51 | CH | 0 | CF₃ | (CH₂)₂ | ″ | |
| 52 | CH | 0 | CF₃ | (CH₂)₃ | ″ | |
| 53 | CH | 0 | CF₃ | CH₂ | 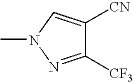 | 69-70 |
| 54 | CH | 0 | CF₃ | (CH₂)₂ | ″ | |
| 55 | CH | 0 | CF₃ | (CH₂)₃ | 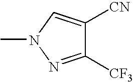 | |
| 56 | CH | 0 | CF₃ | CH₂ | 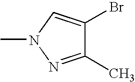 | oil |
| 57 | CH | 0 | CF₃ | (CH₂)₂ | ″ | |
| 58 | CH | 0 | CF₃ | (CH₂)₃ | ″ | |
| 59 | CH | 0 | CF₃ | CH₂ | 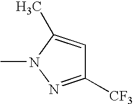 | 82-85 |
| 60 | CH | 0 | CF₃ | (CH₂)₂ | ″ | |
| 61 | CH | 0 | CF₃ | (CH₂)₃ | ″ | |
| 62 | CH | 0 | CF₃ | (CH₂) | 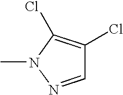 | oil |
| 63 | CH | 0 | CF₃ | (CH₂)₃ | ″ | |
| 64 | CH | 0 | CF₃ | (CH₂)₃ | ″ | |
| 65 | CH | 0 | CF₃ | CH₂ | 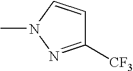 | 78-79 |
| 66 | CH | 0 | CF₃ | (CH₂)₂ | ″ | |
| 67 | CH | 0 | CF₃ | (CH₂)₃ | ″ | |
| 68 | CH | 0 | CF₃ | (CH₂)₃ | 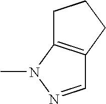 | |
| 69 | CH | 0 | CF₃ | (CH₂)₃ | ″ | |
| 70 | CH | 0 | CF₃ | (CH₂)₃ | ″ | |
| 71 | CH | 0 | CF₃ | (CH₂)₃ | 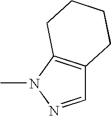 | |
| 72 | CH | 0 | CF₃ | (CH₂)₃ | ″ | oil |
| 73 | CH | 9 | CF₃ | (CH₂)₃ | ″ | |
| 74 | CH | 0 | CF₃ | CH₂ | 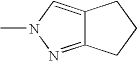 | |
| 75 | CH | 0 | CF₃ | (CH₂)₂ | ″ | |
| 76 | CH | 0 | CF₃ | (CH₂)₃ | ″ | |
| 77 | CH | 0 | CF₃ | CH₂ | 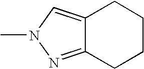 | |
| 78 | CH | 0 | CF₃ | (CH₂)₂ | ″ | |
| 79 | CH | 0 | CF₃ | (CH₂)₃ | ″ | |
| 80 | CH | 0 | CF₃ | CH₂ | 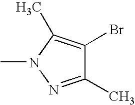 | 107-109 |
| 81 | CH | 0 | CF₃ | (CH₂)₂ | ″ | |
| 82 | CH | 0 | CF₃ | (CH₂)₃ | ″ | |
| 83 | CH | 0 | CF₃ | CH₂ | 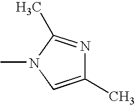 | |
| 84 | CH | 0 | CF₃ | (CH₂)₂ | ″ | |
| 85 | CH | 0 | CF₃ | (CH₂)₃ | ″ | |
| 86 | CH | 0 | CF₃ | CH₂ | 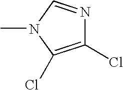 | |
| 87 | CH | 0 | CF₃ | (CH₂)₂ | ″ | |
| 88 | CH | 0 | CF₃ | (CH₂)₃ | ″ | |
| 89 | CH | 0 | CF₃ | CH₂ | 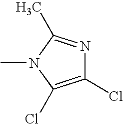 | 117-120 |
| 90 | CH | 0 | CF₃ | (CH₂)₂ | ″ | |
| 91 | CH | 0 | CF₃ | (CH₂)₃ | 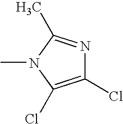 | |
| 92 | CH | 0 | CF₃ | CH₂ | 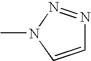 | oil |
| 93 | CH | 0 | CF₃ | (CH₂)₂ | ″ | |
| 94 | CH | 0 | CF₃ | (CH₂)₃ | ″ | |
| 95 | CH | 0 | CF₃ | CH₂ | 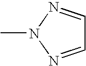 | oil |
| 96 | CH | 0 | CF₃ | (CH₂)₂ | ″ | |
| 97 | CH | 0 | CF₃ | (CH₂)₃ | ″ | |
| 98 | CH | 0 | CF₃ | CH₂ | 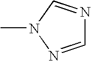 | oil |
| 99 | CH | 0 | CF₃ | (CH₂)₂ | ″ | 42-43 |
| 100 | CH | 0 | CF₃ | (CH₂)₃ | ″ | |
| 101 | CH | 0 | CF₃ | (CH₂)₄ | ″ | |
| 102 | CH | 0 | CF₃ | (CH₂)₅ | ″ | |
| 103 | CH | 0 | CF₃ | (CH₂)₈ | ″ | |
| 104 | CH | 0 | CF₃ | CH₂CH(CH₃) | ″ | oil |
| 105 | N | 0 | CF₃ | (CH₂) | ″ | |
| 106 | N | 0 | CF₃ | (CH₂)₂ | ″ | |
| 107 | CH | 1 | CF₃ | (CH₂)₂ | ″ | |
| 108 | CH | 0 | CF₂ | (CH₂)₂ | ″ | |
| | | | Cl | | | |
| 109 | CH | 0 | CF₃ | CH₂ | 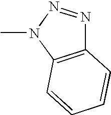 | |
| 110 | CH | 0 | CF₃ | (CH₂)₂ | ″ | 102-103 |
| 111 | CH | 0 | CF₃ | (CH₂)₃ | 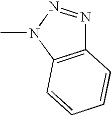 | |
| 112 | CH | 0 | CF₃ | CH₂ | 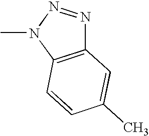 | |
| 113 | CH | 0 | CF₃ | (CH₂)₂ | ″ | 105-106 |
| 114 | CH | 0 | CF₃ | (CH₂)₃ | ″ | |
| 115 | CH | 0 | CF₃ | CH₂ | 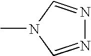 | |
| 116 | CH | 0 | CF₃ | (CH₂)₂ | ″ | |
| 117 | CH | 0 | CF₃ | (CH₂)₃ | ″ | |
| 118 | CH | 0 | CF₃ | CH₂ | 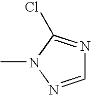 | |
| 119 | CH | 0 | CF₃ | (CH₂)₂ | ″ | |
| 120 | CH | 0 | CF₃ | (CH₂)₃ | ″ | |
| 121 | CH | 0 | CF₃ | CH₂ | 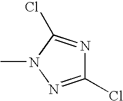 | |
| 122 | CH | 0 | CF₃ | (CH₂)₂ | ″ | |
| 123 | CH | 0 | CF₃ | (CH₂)₃ | ″ | |
| 124 | CH | 0 | CF₃ | CH₂ | 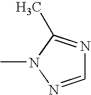 | |
| 125 | CH | 0 | CF₃ | (CH₂)₂ | ″ | |
| 126 | CH | 0 | CF₃ | (CH₂)₃ | ″ | |
| 127 | CH | 0 | CF₃ | CH₂ | 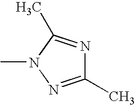 | |
| 128 | CH | 0 | CF₃ | (CH₂)₂ | ″ | |
| 129 | CH | 0 | CF₃ | (CH₂)₃ | ″ | |
| 130 | CH | 0 | CF₃ | CH₂ | 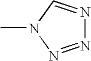 | |
| 131 | CH | 0 | CF₃ | (CH₂)₂ | ″ | |
| 132 | CH | 0 | CF₃ | (CH₂)₃ | ″ | |
| 133 | CH | 0 | CF₃ | CH₂ | 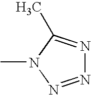 | |
| 134 | CH | 0 | CF₃ | (CH₂) | ″ | |
| 135 | CH | 0 | CF₃ | (CH₂)₃ | ″ | |
| 136 | CH | 0 | CF₃ | (CH₂)₃ | 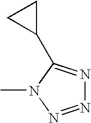 | |
| 137 | CH | 0 | CF₃ | (CH₂)₃ | ″ | |
| 138 | CH | 0 | CF₃ | (CH₂)₃ | ″ | |
| 139 | CH | 0 | CF₃ | CH₂ | 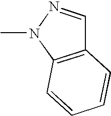 | |
| 140 | CH | 0 | CF₃ | (CH₂)₂ | ″ | oil |
| 141 | CH | 0 | CF₃ | (CH₂)₃ | 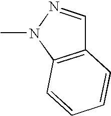 | |
| 142 | CH | 0 | CF₃ | CH₂ | 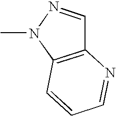 | |
| 143 | CH | 0 | CF₃ | (CH₂)₂ | ″ | |
| 144 | CH | 0 | CF₃ | (CH₂)₃ | ″ | |
| 145 | CH | 0 | CF₃ | CH₂ | 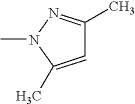 | |
| 146 | CH | 0 | CF₃ | (CH₂)₂ | ″ | 80-81 |
| 147 | CH | 0 | CF₃ | (CH₂)₃ | ″ | |
| 148 | CH | 0 | CF₃ | CH₂ | 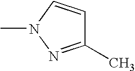 | |
| 149 | CH | 0 | CF₃ | (CH₂)₂ | ″ | |
| 150 | CH | 0 | CF₃ | (CH₂)₃ | ″ | |
| 151 | CH | 0 | CF₃ | CH₂ | 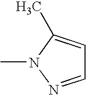 | |
| 152 | CH | 0 | CF₃ | CH₂ | ″ | |
| 153 | CH | 0 | CF₃ | CH₂ | ″ | |
| 154 | CH | 0 | CF₃ | CH₂ | 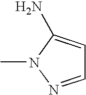 | |
| 155 | CH | 0 | CF₃ | CH₂ | ″ | |
| 156 | CH | 0 | CF₃ | CH₂ | ″ | |
| 157 | CH | 0 | CF₃ | CH₂ | 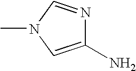 | |
| 158 | CH | 0 | CF₃ | CH₂ | ″ | |
| 159 | CH | 0 | CF₃ | CH₂ | 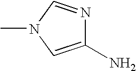 | |
| 160 | CH | 0 | CF₃ | CH₂ | 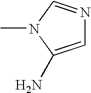 | |
| 161 | CH | 0 | CF₃ | CH₂ | ″ | |
| 162 | CH | 0 | CF₃ | CH₂ | ″ | |
| 163 | CH | 0 | CF₃ | CH₂ | 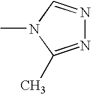 | |
| 164 | CH | 0 | CF₃ | CH₂ | ″ | |
| 165 | CH | 0 | CF₃ | CH₂ | ″ | |
| 166 | CH | 0 | CF₃ | CH₂ | 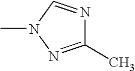 | |
| 167 | CH | 0 | CF₃ | CH₂ | ″ | |
| 168 | CH | 0 | CF₃ | CH₂ | ″ | |
| 169 | CH | 0 | CF₃ | CH₂ | 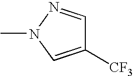 | |
| 170 | CH | 0 | CF₃ | CH₂ | ″ | |
| 171 | CH | 0 | CF₃ | CH₂ | ″ | |
| 172 | CH | 0 | CF₃ | CH₂ | 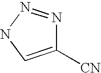 | |
| 173 | CH | 0 | CF₃ | (CH₂)₂ | 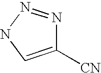 | |
| 174 | CH | 0 | CF₃ | (CH₂)₃ | ″ | |
| 175 | CH | 0 | CF₃ | CH₂ | 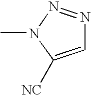 | |
| 176 | CH | 0 | CF₃ | (CH₂)₂ | ″ | |
| 177 | CH | 0 | CF₃ | (CH₂)₃ | ″ | |
| 178 | CH | 0 | CF₃ | CH₂ | 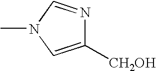 | |
| 179 | CH | 0 | CF₃ | (CH₂)₂ | ″ | |
| 180 | CH | 0 | CF₃ | (CH₂)₃ | ″ | |
| 181 | CH | 0 | CF₃ | CH₂ | 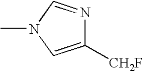 | |
| 182 | CH | 0 | CF₃ | (CH₂)₂ | ″ | |
| 183 | CH | 0 | CF₃ | (CH₂)₃ | ″ | |
| 184 | CH | 0 | CF₃ | CH₂ | 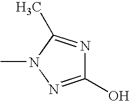 | |
| 185 | CH | 0 | CF₃ | (CH₂)₂ | ″ | |
| 186 | CH | 0 | CF₃ | (CH₂)₃ | ″ | |
| 187 | CH | 0 | CF₃ | CH₂ | 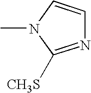 | |
| 188 | CH | 0 | CF₃ | (CH₂)₂ | 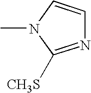 | |
| 189 | CH | 0 | CF₃ | (CH₂)₃ | ″ | |
| 190 | CH | 0 | CF₃ | CH₂ | 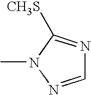 | |
| 191 | CH | 0 | CF₃ | (CH₂)₂ | ″ | |
| 192 | CH | 0 | CF₃ | (CH₂)₃ | ″ | |
| 193 | CH | 0 | CF₃ | CH₂ | 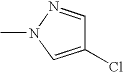 | |
| 194 | CH | 0 | CF₃ | (CH₂)₂ | ″ | |
| 195 | CH | 0 | CF₃ | (CH₂)₃ | ″ | |
| 196 | CH | 0 | CF₃ | CH₂ | 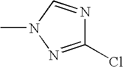 | |
| 197 | CH | 0 | CF₃ | (CH₂)₂ | ″ | |
| 198 | CH | 0 | CF₃ | (CH₂)₃ | ″ | |
| 199 | CH | 0 | CF₃ | CH₂ | 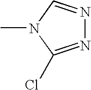 | |
| 200 | CH | 0 | CF₃ | (CH₂)₂ | ″ | |
| 201 | CH | 0 | CF₃ | (CH₂)₃ | ″ | |
| 202 | CH | 0 | CF₃ | CH₂ | 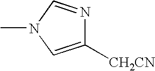 | |
| 203 | CH | 0 | CF₃ | (CH₂)₂ | ″ | |
| 204 | CH | 0 | CF₃ | (CH₂)₃ | 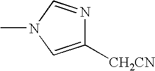 | |
| 205 | CH | 0 | CF₃ | CH₂ | 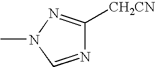 | |
| 206 | CH | 0 | CF₃ | (CH₂)₂ | 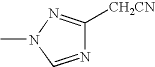 | |
| 207 | CH | 0 | CF₃ | (CH₂)₃ | ″ | |
| 208 | CH | 0 | CF₃ | CH₂ | 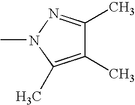 | |
| 209 | CH | 0 | CF₃ | (CH₂)₂ | ″ | |
| 210 | CH | 0 | CF₃ | (CH₂)₃ | ″ | |
| 211 | CH | 0 | CF₃ | CH₂ | 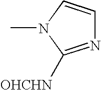 | |
| 212 | CH | 0 | CF₃ | (CH₂)₂ | ″ | |
| 213 | CH | 0 | CF₃ | (CH₂)₃ | ″ | |
| 214 | CH | 0 | CF₃ | CH₂ | 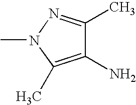 | |
| 215 | CH | 0 | CF₃ | (CH₂)₂ | ″ | |
| 216 | CH | 0 | CF₃ | (CH₂)₃ | ″ | |
| 217 | CH | 0 | CF₃ | CH₂ | 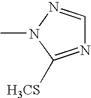 | |
| 218 | CH | 0 | CF₃ | (CH₂)₂ | ″ | |
| 219 | CH | 0 | CF₃ | (CH₂)₃ | ″ | |
| 220 | CH | 0 | CF₃ | CH₂ | 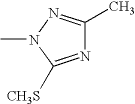 | |
| 221 | CH | 0 | CF₃ | (CH₂)₂ | ″ | |
| 222 | CH | 0 | CF₃ | (CH₂)₃ | ″ | |
| 223 | CH | 0 | CF₃ | CH₂ | 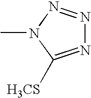 | |
| 224 | CH | 0 | CF₃ | (CH₂)₂ | ″ | |
| 225 | CH | 0 | CF₃ | (CH₂)₃ | ″ | |
| 226 | CH | 0 | CF₃ | CH₂ | 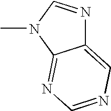 | |
| 227 | CH | 0 | CF₃ | (CH₂)₂ | ″ | |
| 228 | CH | 0 | CF₃ | (CH₂)₃ | ″ | |
| 229 | CH | 0 | CF₃ | CH₂ | 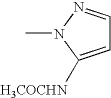 | |
| 230 | CH | 0 | CF₃ | (CH₂)₂ | ″ | |
| 231 | CH | 0 | CF₃ | (CH₂)₃ | ″ | |
| 232 | CH | 0 | CF₃ | CH₂ | 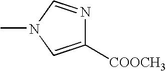 | |
| 233 | CH | 0 | CF₃ | (CH₂)₂ | ″ | |
| 234 | CH | 0 | CF₃ | (CH₂)₃ | ″ | |
| 235 | CH | 0 | CF₃ | CH₂ | 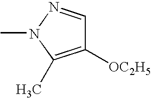 | |
| 236 | CH | 0 | CF₃ | (CH₂)₂ | ″ | |
| 237 | CH | 0 | CF₃ | (CH₂)₃ | ″ | |
| 238 | CH | 0 | CF₃ | CH₂ | 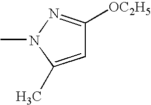 | |
| 239 | CH | 0 | CF₃ | (CH₂)₂ | ″ | |
| 240 | CH | 0 | CF₃ | (CH₂)₃ | ″ | |
| 241 | CH | 0 | CF₃ | CH₂ | 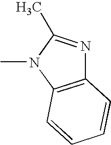 | |
| 242 | CH | 0 | CF₃ | (CH₂)₂ | ″ | 93-95 |
| 243 | CH | 0 | CF₃ | (CH₂)₃ | ″ | |
| 244 | CH | 0 | CF₃ | CH₂ | 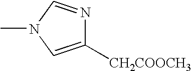 | |
| 245 | CH | 0 | CF₃ | (CH₂)₂ | ″ | |
| 246 | CH | 0 | CF₃ | (CH₂)₃ | ″ | |
| 247 | CH | 0 | CF₃ | CH₂ | 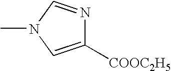 | |
| 248 | CH | 0 | CF₃ | (CH₂)₂ | ″ | |
| 249 | CH | 0 | CF₃ | (CH₂)₃ | ″ | |
| 250 | CH | 0 | CF₃ | CH₂ | 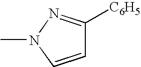 | |
| 251 | CH | 0 | CF₃ | CH₂ | ″ | |
| 252 | CH | 0 | CF₃ | CH₂ | ″ | |
| 253 | CH | 0 | CF₃ | CH₂ | 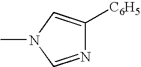 | |
| 254 | CH | 0 | CF₃ | CH₂ | ″ | |
| 255 | CH | 0 | CF₃ | CH₂ | ″ | |
| 256 | CH | 0 | CF₃ | CH(CH₃) | 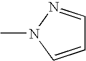 | oil |
| 257 | CH | 0 | CF₃ | CH₂CH(CH₃)CH₂ | ″ | |
| 258 | CH | 0 | CF₃ | CH(CH₃)CH₂ | ″ | |
| 259 | CH | 0 | CF₃ | CH(CH₃) | 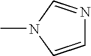 | |
| 260 | CH | 0 | CF₃ | CH₂CH(CH₃)CH₂ | ″ | |
| 261 | CH | 0 | CF₃ | CH(CH₃)CH₂ | ″ | |
| 262 | CH | 0 | CF₃ | CH(CH₃) | 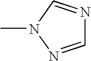 | |
| 263 | CH | 0 | CF₃ | CH₂CH(CH₃)CH₂ | ″ | |
| 264 | CH | 0 | CF₃ | CH(CH₃)CH₂ | ″ | 74-75 |
| 265 | CH | 0 | CF₃ | CH₂C(CH₃)₂ | ″ | 71-73 |
| 266 | CH | 0 | CF₃ | CH₂ | 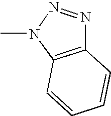 | |
| 267 | CH | 0 | CF₃ | (CH₂)₂ | 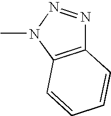 | 128-129 |
| 268 | CH | 0 | CF₃ | (CH₂)₃ | ″ | |
| 269 | CH | 0 | CF₃ | CH₂ | 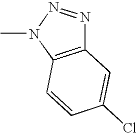 | |
| 270 | CH | 0 | CF₃ | (CH₂)₂ | ″ | 122-123 |
| 271 | CH | 0 | CF₃ | (CH₂)₃ | ″ | |
| 272 | CH | 0 | CF₃ | CH₂ | 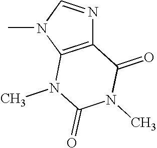 | |
| 273 | CH | 0 | CF₃ | (CH₂)₂ | ″ | 161-162 |
| 274 | CH | 0 | CF₃ | (CH₂)₃ | ″ | |
| 275 | CH | 0 | CF₃ | CH₂ | 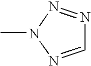 | |
| 276 | CH | 0 | CF₃ | (CH₂)₂ | ″ | |
| 277 | CH | 0 | CF₃ | (CH₂)₃ | ″ | |
| 278 | CH | 0 | CF₃ | CH₂ | 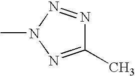 | |
| 279 | CH | 0 | CF₃ | (CH₂)₂ | ″ | |
| 280 | CH | 0 | CF₃ | (CH₂)₃ | ″ | |
| 281 | CH | 0 | CF₃ | CH₂ | 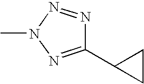 | |
| 282 | CH | 0 | CF₃ | (CH₂)₂ | ″ | |
| 283 | CH | 0 | CF₃ | (CH₂)₃ | ″ | |
| 284 | CH | 0 | 0 | CH₂ | 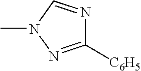 | |
| 285 | CH | 0 | 0 | (CH₂)₂ | ″ | |
| 286 | CH | 0 | 0 | (CH₂)₃ | ″ | |

TABLE 2

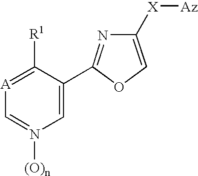

| Ex. No. | A | N | R¹ | X | Az | m.p. [° C.] |
|---|---|---|---|---|---|---|
| 300 | CH | 0 | CF₃ | CH₂ | 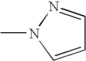 | |
| 301 | CH | 0 | CF₃ | (CH₂)₂ | ″ | |
| 302 | CH | 0 | CF₃ | (CH₂)₃ | ″ | |
| 303 | CH | 0 | CF₃ | CH₂ | 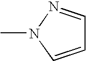 | oil |
| 304 | CH | 0 | CF₃ | (CH₂)₂ | 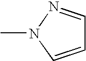 | |
| 305 | CH | 0 | CF₃ | (CH₂)₃ | ″ | |
| 306 | CH | 0 | CF₃ | CH₂ | 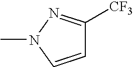 | |
| 307 | CH | 0 | CF₃ | (CH₂)₂ | ″ | |
| 308 | CH | 0 | CF₃ | (CH₂)₃ | ″ | |
| 309 | CH | 0 | CF₃ | CH₂ | 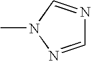 | |
| 310 | CH | 0 | CF₃ | (CH₂)₂ | ″ | |
| 311 | CH | 0 | CF₃ | (CH₂)₃ | ″ | |
| 312 | CH | 0 | CF₃ | CH₂ | 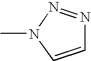 | |
| 313 | CH | 0 | CF₃ | (CH₂)₂ | ″ | |
| 314 | CH | 0 | CF₃ | (CH₂)₃ | ″ | |

TABLE 3

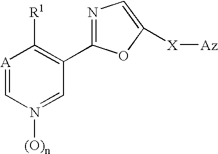

| Ex. No. | A | N | R¹ | X | Az | m.p. [° C.] |
|---|---|---|---|---|---|---|
| 400 | CH | 0 | CF₃ | CH₂ | 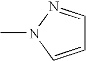 | |
| 401 | CH | 0 | CF₃ | (CH₂)₂ | ″ | |
| 402 | CH | 0 | CF₃ | (CH₂)₃ | ″ | |
| 403 | CH | 0 | CF₃ | CH₂ | 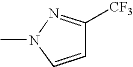 | |
| 404 | CH | 0 | CF₃ | (CH₂)₂ | ″ | |
| 405 | CH | 0 | CF₃ | (CH₂)₃ | ″ | |
| 406 | CH | 0 | CF₃ | CH₂ | 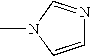 | |
| 407 | CH | 0 | CF₃ | (CH₂)₂ | ″ | |
| 408 | CH | 0 | CF₃ | (CH₂)₃ | ″ | |
| 409 | CH | 0 | CF₃ | CH₂ | 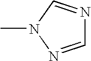 | |
| 410 | CH | 0 | CF₃ | (CH₂)₂ | ″ | |
| 411 | CH | 0 | CF₃ | (CH₂)₃ | ″ | |
| 412 | CH | 0 | CF₃ | CH₂ | 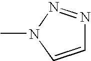 | |
| 413 | CH | 0 | CF₃ | (CH₂)₂ | ″ | |
| 414 | CH | 0 | CF₃ | (CH₂)₃ | ″ | |

### B. FORMULATION EXAMPLES

a) A dust is obtained by mixing 10 parts by weight of active substance and 90 parts by weight of talc as inert substance and comminuting the mixture in a hammermill.

b) A wettable powder which is readily dispersible in water is obtained by mixing 25 parts by weight of active substance, 65 parts by weight of kaolin-containing quartz as inert substance, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is prepared by mixing 40 parts by weight of active substance with 7 parts by weight of a sulfosuccinic aminoester, 2 parts by weight of a sodium lignosulfonate and 51 parts by weight of water and grinding the mixture in a ball mill to a fineness of below 5 microns.

d) An emulsifiable concentrate can be prepared from 15 parts by weight of active substance, 75 parts by weight of cyclohexane as solvent and 10 parts by weight of oxethylated nonylphenol (10 EO) as emulsifier.

e) Granules can be prepared from 2 to 15 parts by weight of active substance and an inert granule carrier material such as attapulgite, pumice granules and/or quartz sand. It is expedient to use a suspension of the wettable powder of Example b) with a solids content of 30% and spray this onto the surface of attapulgite granules, dry the material and mix it intimately. The wettable powder amounts to approximately 5% by weight and the inert carrier material to approximately 95% by weight of the finished granules.

### C. BIOLOGICAL EXAMPLES

### Example 1

Pregerminated field bean seeds (Vicia faba) with seminal roots were transferred into amber glass bottles filled with tap water and then populated with approximately 100 black bean aphids (Aphis fabae). The plants and aphids were then immersed for 5 seconds in an aqueous solution of the formulated preparation to be tested. After the solution had been allowed to run off, the plants and animals were stored in a controlled-environment cabinet (16 hours light/day, 25° C., 40-60% atmospheric humidity). After storage for 3 and 6 days, the effect of the preparation on the aphids was determined. At a concentration of 300 ppm (based on the active substance content), the preparations of Examples No. 1, 13, 15, 18, 27, 30, 41, 44, 47, 50, 53, 56, 59, 62, 65, 80, 89, 92, 95, 98, 99, 113, 242, 273 and 303 resulted in 90-100% mortality of the aphids.

### Example 2

The leaves of 12 rice plants with a stem length of 8 cm were immersed for 5 seconds in an aqueous solution of the formulated preparation to be tested. After the solution had been allowed to run off, the rice plants treated thus were placed into a Petri dish and populated with approximately 20 larvae (L3 instar) of the rice leafhopper species Nilaparvata lugens. The Petri dish was sealed and then stored in a controlled-environment cabinet (16 hours light/day, 25° C., 40-60% atmospheric humidity). After storage for 6 days, the mortality of the leafhopper larvae was determined. At a concentration of 300 ppm (based on active substance content), the preparations of Examples No. 27, 50, 53, 56, 59, 62, 80, 98, 99, 113, 242 and 273 resulted in 90-100% mortality.

### Example 3

Pregerminated field bean seeds (Vicia faba) with seminal roots were transferred into amber glass bottles filled with tap water. Four milliliters of an aqueous solution of the formulated preparation to be tested were pipetted into the amber glass bottle. Then, the field bean was infested with approx. 100 black bean aphids (Aphis fabae). The plants and animals were then stored in a controlled-environment cabinet (16 hours light/day, 25° C., 40-60% atmospheric humidity). After storage for 3 and 6 days, the root-systemic action of the preparation on the aphids was determined. At a concentration of 300 ppm (based on active substance content), the preparations of Examples No. 1, 13, 15, 18, 27, 30, 41, 44, 47, 50, 53, 56, 59, 65, 80, 89, 92, 95, 98, 99 and 303 caused 90-100% mortality of the aphids owing to the root-systemic activity.

We claim:

1. An azolylalkyloxazole or -oxadiazole derivative of the formula (I) 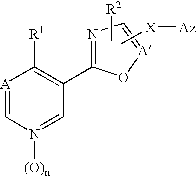
where the symbols and indices are defined as follows:
R¹ is (C₁-C₄)haloalkyl;
R² is hydrogen, halogen, (C₁-C₄)alkyl, (C₃-C₈)cycloalkyl or (C₁-C₄)haloalkyl;
A, A′ are identical or different and are in each case CH or N;
n is 0 or 1;
X is a branched or unbranched (C₁-C₈)alkylene unit in which a C—C single bond can optionally be replaced by a double or triple bond and in which furthermore one or more CH₂ groups can be replaced by a carbonyl group or a heteroatom unit and where additionally 3 to 8 atoms of this hydrocarbon radical, which is optionally modified as above, may form a cycle;
Az is a group of the formula (II) 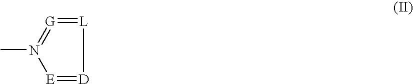
where the symbols have the following meanings:
E, D, G, L are identical or different and are CH or N, where in each case at least one of the symbols E, D, G and L must be CH and at least one must be N
and where carbon atoms are optionally substituted and the substituents of adjacent carbon atoms, optionally together with the carbon atoms of the group Az, can form a ring.

2. A compound of the formula (I) as claimed in claim 1, where
R¹ is (C₁-C₄)fluoroalkyl,
R² is hydrogen or methyl,
n is 0 and
A is CH.

3. A compound of the formula (I) as claimed in claim 2, where
R¹ is trifluoromethyl,
R² is hydrogen,
n is 0 and
A is CH.

4. A compound of the formula (I) as claimed in claim 3, where
R¹ if trifluoromethyl,
n is 0,
A is CH and
A′ is nitrogen.

5. A compound of the formula (I) as claimed in claim 1, where
Az is an imidazolyl, pyrazolyl or 1,2,4-triazol-1-yl radical and is optionally substituted by one or more radicals R⁴, and
R⁴ is (C₁-C₄)alkyl, trifluoromethyl, fluorine, chlorine, bromine, cyano or nitro, or two radicals R⁴ are linked to give a benzo fusion.

6. A compound of the formula (I) as claimed in claim 5, where
Az is a pyrazolyl or 1,2,4-triazol-1-yl radical and, if appropriate,
R⁴ is methyl, chlorine, bromine, cyano, nitro or trifluoromethyl.

7. A process for the preparation of a compound of the formula (I) where A, R¹, X, n and Az are as defined for formula (I) in claim 1 and A′ is nitrogen, which comprises
reacting a compound of the formula (III), 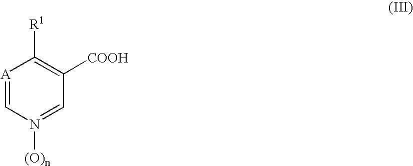
in which A, R¹ and n are as defined in formula (I) in claim 1, if appropriate in the form of an activated derivative of this acid, in the presence of a base with a compound of the formula (IV) 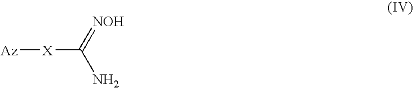
in which X and Az are as defined in formula (I) in claim 1.

8. A process for the preparation of a compound of the formula (I) where A, R¹, X, n and Az are as defined for formula (I) in claim 1 and A′ is CH, which comprises reacting a compound of the formula (VI) 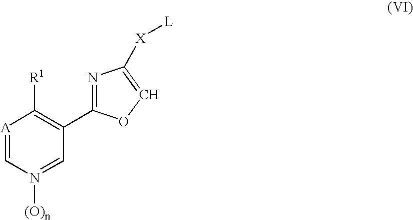
in which A, R¹, n and X are as defined for formula (I) in claim 1 and L is a leaving group
with an optionally substituted azole of the formula (VII) 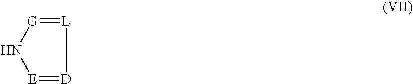
in which D, E, G, L and m are as defined for formula (II) in claim 1.

9. An insecticidally and/or acaricidally active composition comprising at least one compound as claimed in claim 1.

10. An insecticidally and/or acaricidally active composition as claimed in claim 9 in a mixture with carriers and/or surfactants.

11. A composition as claimed in claim 9 which comprises an additional active substance from the group of the acaricides, fungicides, herbicides, insecticides, nematicides or growth regulators.

12. A composition for treating animals comprising a compound of the formula (I) as claimed in claim 1.

13. A method of controlling harmful insects and/or Acarina, which comprises applying an effective amount of a compound as claimed in claim 1 to the site where the action is desired.

14 A method of controlling harmful insects and/or Acarina, which comprises applying an effective amount of a composition as claimed in claim 9 to the site where the action is desired

15. A method of protecting useful plants against the undesired effects of harmful insects and/or Acarina, which comprises treating the seeds of the useful plants with a compound as claimed in claim 1.

16. A method of protecting useful plants against the undesired effects of harmful insects and/or Acarina, which comprises treating the seeds of the useful plants with a compound as claimed in claim 9.
